(19) United States
(12) Patent Application Publication
(10) Pub. No.: US 2005/0233394 A1
(43) Pub. Date: Oct. 20, 2005

(54) Serological diagnostic method comprising positive control of introduction of human serum to be tested

(76) Inventor: Didier M Raoult, Marseille (FR)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/519,455

(22) PCT Filed: Jul. 2, 2003
(86) PCT No.: PCT/FR03/02050

§ 371 Date: Dec. 29, 2004

(30) Foreign Application Priority Data
Jul. 3, 2002 (FR) 02/08324

(51) Int. Cl. G01N 33/554; G01N 33/569

(52) U.S. Cl. 435/7.32

(57) ABSTRACT

The invention concerns the use of an antigen containing protein A, and in particular of Staphylococcus aureus as non-specific antigen added to bacterial serology reactions as positive control that the serum to be tested has been included.

The present invention concerns a diagnosis method for human infectious diseases. More particularly, it concerns the area of indirect diagnosis via serology. One laboratory method for diagnosing human infectious diseases is microbial serology. It is an indirect diagnosis method based on the search in a patient's serum for specific antibodies against the microbial agent, namely a bacterium, a virus, a parasite or a fungus responsible for a pathology (hereinafter called “microbe”). This diagnosis method is essential for diagnosing infections with culture micro-organisms that are difficult to identify, in particular viruses, strict intracellular bacteria and facultative intracellular bacteria of the-genus Rickettsia, Coxiella, Bartonella, Tropheryma, Ehrlichia, Chlamydia, Mycoplasma, Treponema, Borrelia, and Leptospira for example.

Technically, microbial serology consists of detecting, in the patient's serum, an antigen-antibody reaction in which the antigen is represented by all or part of the infectious microbial agent to be detected, and the antibody is represented by human immunoglobulins specific to said infectious microbial agent that are present in the patient's serum. It may be quantified by successively testing a series of increasing twofold or tenfold dilutions of the patient's serum.

Conventionally, a serological diagnosis method more particularly comprises the following steps:
1. Depositing the antigen on a solid substrate such as latex micro-beads, in particular when using the agglutination detection technique in which latex micro-beads are coated with the microbial antigen to be tested and agglutinate if the patient's serum carries specific antibodies, this agglutination being visible to the naked eye; or on a glass slide or filtration microplate for detection methods by immunofluorescence or enzyme technique, in particular of ELISA type (“Enzyme Linked Immunosorbent Assay”), or on nitrocellulose or nylon paper for detection techniques of Western-blot type, in which the microbial antigens separated by electrophoresis then transferred onto a solid substrate (nitrocellulose or nylon paper) react separately from one another with the patient's serum. These separation techniques are well known to persons skilled in the art;
2. Serum decomplementation by heating to 56° C. for 30 minutes;
3. Contacting the antigen corresponding to the infectious microbial agent with the patient's serum, then incubating under conditions of time, temperature, hygrometry, mechanical stirring and ionic strength of the medium which allow the antigen-antibody reaction;
4. Careful, extensive washing to remove the excess patient serum that has not attached to the solid substrate;
5. Applying a secondary detection antibody which is an anti-human immunoglobulin, in particular a goat immunoglobulin conjugated with a fluorochromous substance, generally fluorescein isothiosulphate or an enzyme, generally a peroxidase, and incubating under conditions of time, temperature, hygrometry, mechanical stirring and ionic strength of the medium enabling the antigen-antibody reaction;
6. Careful, extensive washing to remove the excess, non-fixed conjugated immunoglobulin;
7. Detection of a reaction by reading an apparatus such as a fluorescence microscope for the indirect immunofluorescence technique, or optical density reading for an enzyme detection technique of ELISA type. Reading of the Western-Blot reaction can be made with the naked eye, or after digital loading of the gel and analysis using densitometry software.

Each of these steps may be the source of handling errors resulting in the absence of an antigen-antibody reaction, and hence in a false negative result. A handling error, therefore, may lead to interpreting as negative a reaction that is in fact positive (false-negative). A frequent error in serological testing, in particular in battery serological tests made on a large number of sera to be tested, is due to faulty addition of the sera to be tested, in particular by pipetting. These errors especially occur during those steps involving the transfer, in particular by pipetting, of the sample to be tested, since some containers, in particular those containing the solid substrate on which the antigen to be detected is deposited, may inadvertently fail to be filled with the human serum to be tested. It is known that serum pipetting entails an error risk of 1% o, that is related to a purely technical problem of failed pipetting by the pipette or a human error through inadvertent non-pipetting.

These errors require the use of controls when conducting the reaction. The systematic incorporation for each new handling operation of a negative control serum i.e. not containing antibodies specific to the antigen to be tested, makes it possible to interpret positive reactions. Similarly, the incorporation of a positive control serum i.e. containing the antibody specific to the antigen tested at a known titre, makes it possible to verify the quality of the antigen and conjugated immunoglobulin.

However, at the present time no control exists to verify that the serum to be tested has truly been added to the serological test. Yet, if through inadvertence, the serum to be tested is not added to the serological test, the bacterial antigen-serum antibody reaction will certainly not take place and the test will wrongly be interpreted as negative (false-negative).

Protein A is a polypeptide of 42 kDa, and is one of the constituents of the wall of Staphylococcus aureus strains, similar but different proteins are characterized on the surface of bacteria of the genus Streptococcus [Langone J. J. Adv. Immunol. 1982, 32: 157-251].

It is known that a fraction of the wall of Staphylococcus aureus binds to human immunoglobulins, and that protein A is the constituent of the wall of Staphylococcus aureus which binds to human serum [Forsgren A. and J. Sjöquist. J. Immunol. 1966, 97 :822-827], and more particularly at the Fc end of human immunoglobulins. Protein A has four binding sites with the Fc end of immunoglobulins, including two fixation sites which are movable during one same antigen-antibody reaction. Crystalline analysis of this reaction has been conducted [Deisenhofer J. Biochemistry 1981, 20: 2361-2370]. The affinity of protein A for different animal sera has been examined [Kronvall G. Seal U S, Finstad J., Williams R C Jr. J. Immunol. 1970 104: 140-147; Richman D D, Cleueland P H, Oxman M N, Johnson Km. J. Immunol. 1982 128: 2300-2305] and this protein A shows an affinity for sera of animal origin, in particular equine, bovine, pig, rabbit, guinea-pig, mouse; to a lesser extent, hamster rat and sheep. On the other hand, chick and goat sera do not react with protein A.

Staphylococcus aureus has already been added to serological tests as detector antigen for specific antibodies [Ryding U, Esperson F, Söderquist B, Christensson B. Diag. Microbiol. Infect. Dis. 2002, 42: 9-15]. Published studies have shown the lack of specificity of these tests.

The subject of the present invention is the addition of a control detecting the inclusion of the serum to be tested during bacterial serology reactions.

The inventors have discovered by chance during a serological study in patients suffering from endocarditis that the sera of these patients all reacted against Staphylococcus aureus, and they have shown in the present invention that this is a general property, namely that the reaction of a human serum with an antigen containing protein A is not deteriorated by human serum having an infectious pathology, including infectious pathologies which most modify the level and type of secreted immunoglobulins, such as viral diseases involving the H.I.V., C.M.V. or Epstein-Barr viruses. Whilst it was known that protein A binds to every normal human serum, it was not known that this property is maintained in human sera taken from patients suffering from different infectious pathologies. Nor was it known that Staphylococcus aureus could perform this <<carrier>> role for protein A during bacterial serology reactions.

The present invention therefore essentially consists of adding an antigen containing protein A and in particular of adding a Staphylococcus aureus bacterium as control antigen to control that the sample to be tested truly contains a serum of human origin, and to detect an antigen-human immunoglobulin reaction with a specific substance.

The inventors have found that, insofar as protein A reacts with human immunoglobulins in non-specific manner even in the presence of a major infectious pathology, it is possible to use this protein A as positive control to detect the inclusion of a human serum in the sample to be tested, since in addition protein A does not react with certain antibodies.

More precisely, the present invention provides an in vitro serological diagnosis method in which the presence is detected of antibodies specific to an infectious microbial agent in a sample to be tested, characterized in that it is controlled that said sample to be tested does contain a human serum by detecting whether human immunoglobulins react with a protein A-containing antigen of the bacterium Staphylococcus aureus.

More particularly, the following steps are conducted, in which:
the sample to be tested is caused to react with a first antigen (Ag₁) containing protein A, preferably with all or part of a Staphylococcus aureus bacterium containing protein A, and
the presence is detected of an antigen-antibody reaction product (Ag₁-Ac₁) in which the antibody (Ac₁) is a human immunoglobulin, by causing said reaction product to react with a detection substance which is a substance reacting with a human immunoglobulin and not reacting with said first antigen.

By “detection of human immunoglobulin reacting with a protein A-containing antigen”, is meant the capacity to determine a dilution threshold of the sample to be tested containing a human serum, and more particularly a dilution threshold of the human serum to be tested beyond which it is no longer possible to detect said human immunoglobulin having reacted with said protein A-containing antigen, having regard to the detection method used.

In one preferred embodiment, the following steps are conducted in which:
a) said first antigen containing protein A (Ag₁) is deposited on a solid substrate with one or more second antigens (Ag₂) which are characteristic of a microbial infectious agent or agents respectively, and
b) said first (Ag₁) and second (Ag₂) antigens are caused to react with a serum sample to be tested, and
c) it is detected whether a human immunoglobulin (Ac₁) reacts with said first antigen (Ag₁) by causing the reaction product (Ag₁-Ac₁) to react with a secondary detection antibody (Ac₂) which is a labelled anti-human immunoglobin which does not react with protein A.

In one advantageous embodiment, said first antigen is a whole Staphylococcus aureus bacterium containing protein A. Particular use may be made of the Staphylococcus aureus bacteria deposited with public collections such as the bacteria deposited with A.T.C.C. under No29213 and with C.N.C.M. at Institut Pasteur (France) under number 65.8T.

Also, aside from type-strains, any bacterial strain identified as Staphylococcus aureus may be used as antigen Ag₁.

Further advantageously, the presence of said reaction product is detected with an anti-human immunoglobulin which is an immunoglobulin of animal origin, preferably a goat or chick immunoglobulin.

As solid substrate, any device may be used that is suitable for handling cell and bacterial suspensions, such as tubes, glass slides, Bijoux tubes or rigid microtitre plates in polyethylene, polystyrene, polyvinyl chloride or nitrocellulose comprising micro-wells.

As labelling for the anti-human immunoglobulin, advantageously an enzymatic, radioactive or fluorescent type is used, the latter being the preferred type.

The expression <<fluorescent labelling >> means that the antibody has been made fluorescent by coupling or complexing with an appropriate fluorescent agent such as fluorescein isothiocyanate.

The expression <<radioactive labelling >> means that the antibody carries a radioactive isotope allowing its assay by counting its associated radioactivity, the isotope possibly being carried either on a structural element of the antibody, for example constituent tyrosine residues, or on a suitable radical which has been fixed to it.

The expression <<enzymatic labelling >> means that the specific antibody is coupled to or complexed with an enzyme which, associated with the use of appropriate reagents, allows quantitative measurement of this specific antibody.

The substrate and the reagents are chosen so that the end product of the reaction or sequence of reactions caused by the enzyme and using these substances, is:
either a stained or fluorescent substance which diffuses in the liquid medium surrounding the tested sample and is the substance given final measurement either by spectrophotometry or fluorometry respectively, or is visually interpreted, optionally by comparison with a calibrated colour scale,
or an insoluble stained substance which deposits itself on the tested sample and which can be measured either by reflection photometry or can be visually assessed, optionally by comparison with a calibrated colour scale.

When an immunoglobulin made fluorescent is used, the fluorescence associated with the tested sample is directly read on appropriate apparatus.

When a radioactive probe is used, such as iodine 125 for example, the radioactivity associated with the tested sample is counted in a gamma counter using any suitable method, for example after solubilising the cells with an alkaline solution (a sodium hydroxide solution for example) and collecting the solution containing the radioactivity by means of an absorbent buffer.

When an enzyme is used on the specific antibody, the onset of a stained or fluorescent product is obtained by adding a solution containing the enzyme substrate and one or more auxiliary reagents with which it is finally possible to obtain as end product either a stained product soluble in the medium or an insoluble stained product or a soluble fluorescent product as explained above. The light signal given by the samples so treated is then measured using appropriate apparatus in each case: transmission photometer, reflection photometer or fluorimeter respectively. Alternatively, the staining obtained may be assessed visually, optionally with reference to a calibrated scale of stained solutions.

If alkaline phosphatase is used as enzyme, the coupling of this enzyme with the specific antibody is conducted following the method proposed by Boehringer Mannheim-Biochemica. The preferred substrates of this enzyme are paranitrophenylphosphate for fluorometric reading or bromo-5 chloro-4-umbelliferyl phosphate for fluorometric reading or bromo-5 chloro-4 indolyl-6 phosphate to obtain an insoluble stained reaction product. It is also possible to use β-galactosidase as enzyme whose preferred substrates are orthonitrophenyl β-D-galactropyranoside or methyl-4 umbelliferyl β-D-galactopyranoside.

Preferably, it is possible to couple the specific antibodies with the peroxidase. In this case, the coupling method is derived from the one described by M. B. Wilson et P. K. Nakane in: Immunofluorescence and related staining techniques, W. Knapp, K. Kolubar, G. Wicks ed. Elsevier/North Holland. Amsterdam 1978, p. 215-224.

The reagents used to develop the peroxidase conjugated with the specific antibodies contain hydrogen peroxide, a substrate of the enzyme, and a suitable chromogen for example orthophenylenediamine or azino-2-2′ bis (3-ethyl-benzo thiazoline-6-sulfonic acid), or ABTS, to obtain an end reaction product that is stained and soluble in the medium, or diamino-3,3′ benzidine or amino-3 ethyl-9 carbazole or chloro-4 α-naphtol to obtain an insoluble end reaction product, or parahydroxyphenyl propionic acid to obtain a fluorescent reaction product soluble in the medium.

A further embodiment of the invention is the use of immunoglobulin coupled with acetylcholinesterase.

The acetylcholinesterase is coupled to the antibody preferably using a method derived from the one described in French patent no 2 550 799, or a method which schematically includes preparing fragments of the antibody following a known technique, modifying the enzyme by reaction with a suitable heterobifunctional agent and finally coupling of the products so obtained. Other known methods for constructing immunoenzymatic conjugates may also be used in this case.

The development of the enzymatic activity specifically bound to the antigen recognized by the acetylcholinesterase conjugate is preferably conducted following the well known technique which uses acetylthiocholine as substrate for the enzyme and Ellman's reagent or dithio-5,5′ nitro-2 benzoic acid as chromogen, using any variant adapted to the case in hand, for example the one described by Pradelles et al., in Anal. Chem. 1985, 57 :1170-1173.

Further particularly, a series of tests is conducted with increasing dilutions of the sample to be tested and a detection substance is used (Ac₂) which is an immunoglobulin conjugated with a fluorescent substance, and it is verified if a reaction product (Ag₁-Aci-Ac₂) can be detected by fluorescence at a dilution of the sample to be tested that is 1/200 or less, more exactly that is equal to the threshold dilution used to detect the Ag₂ antigen, i.e. the smallest dilution on and after which the serum sample can give rise to detection of a reaction against the specific antigens Ag₂, this threshold dilution being variable in relation to the pathogen and to the detection method and generally lying between 1:4 and 1:200. As mentioned previously, for each detection method a given titre of antibody (Ac₂) can be determined, i.e. a given dilution threshold of the serum to be tested beyond which said antibody (Ac₂) binding to protein A can no longer be detected.

As shown in the following examples, using an immunofluorescence detection method, the demonstration is given that any human serum incorporated in an indirect immunofluorescence reaction at a dilution of 1/200 or less shows a reaction against Staphylococcus aureus, that is to say that the titre of anti-Staphylococcus aureus antibody is at least 1/200.

In one particular embodiment, said infectious microbial agent consisting of said second antigen is chosen from among micro-organisms containing a virus, a parasite or a fungus.

More particularly, said second antigen is an intracellular bacterium and in particular said second antigen is chosen from among the bacteria belonging to the genus Rickettsia, Coxiella, Bartonella, Tropheryma, Ehrlichia, Chlamydia, Mycoplasma, Treponema, Borrelia, and Leptospira, this list not being exhaustive.

Further particularly, said second antigen corresponding to a bacterium is responsible for an endocarditis.

In another embodiment, said second antigen is a viral antigen, in particular a virus chosen from among the H.I.V., C.M.V. or Epstein-Barr viruses.

A further subject of the invention is a diagnosis kit which can be used to implement the method of the invention and contains at least one positive control to detect the inclusion of a human serum in the sample to be tested comprising a said first antigen containing protein A and reagents permitting detection of the presence of a reaction product of said first antigen with a human immunoglobulin.

More particularly, the diagnosis kit comprises:
a solid substrate on which a said first protein A-containing antigen has been deposited, and a said second antigen corresponding to an infectious microbial agent to be detected, and a detection substance to detect a reaction product of said first antigen with a human immunoglobulin comprising a labelled anti-human immunoglobulin which does not react with protein A, preferably an anti-human immunoglobulin which is a goat or chick immunoglobulin labelled with fluorescent marking.

Other characteristics and advantages of the present invention will become apparent in the light of the following examples.

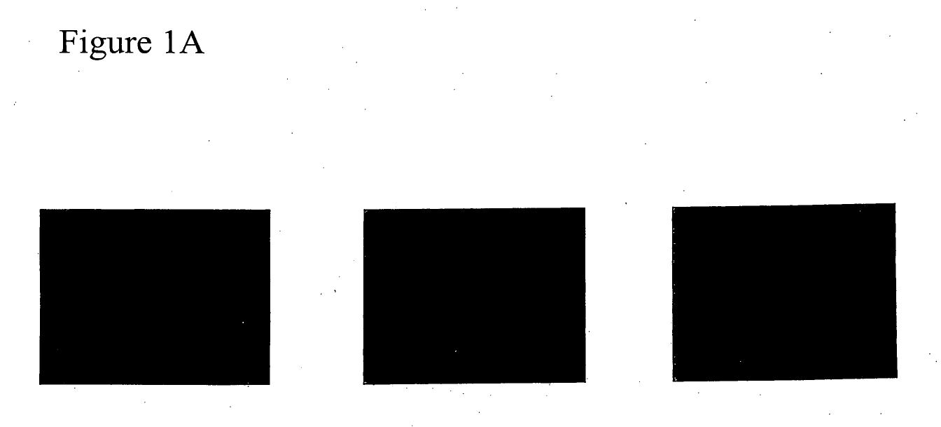

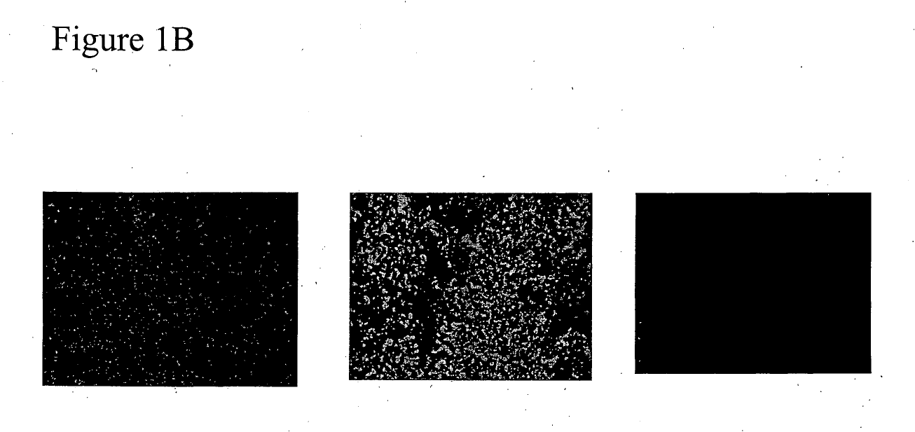

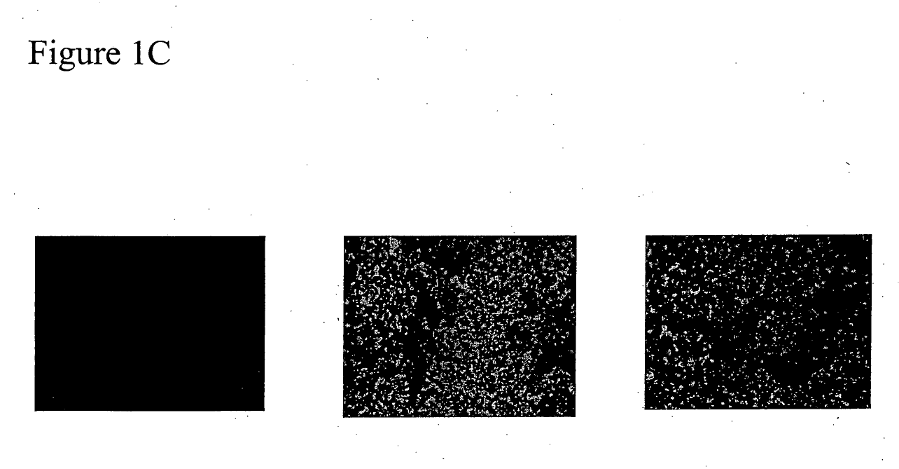

FIGS. 1A, 1B and 1C represent bacterial serological tests conducted using the indirect immunofluorescence method illustrating the invention.

1. Protocol of the Serological Method:

1) Samples

The tested samples comprise:
serum given by a donor person (n=100),
serum from a patient suffering from endocarditis (n=400),
serum from a patient suffering from cat scratch disease due to the bacterium Bartonella henselae, and
serum from a patient having an acute infection with Coxiella burnetti (n=50),
serum from patients suffering from various infections (Epstein-Barr virus, infection H.I.V., C.M.V. infection) (n=50)

The serum is decomplemented by heating to 56° C. for 30 minutes, it may be stored at 4° C. if analysis is not made immediately.

2) Equipment
DINATECH®18-well slide
U-Microplate for dilution réf: Elvetec® 025085096
Fluorescence microscope

3) Reagents
Positive control by antigen
Fluoprep glue réf: Biomérieux 75521. Storage at 20° C.
10 aliquots of antigens: Staphylococcus aureus ATCC 29213, Rickettsia conorii ATCC VR-141, Rickettsia slovaca, Rickettsia typhi ATCC VR-144, Coxiella burnetti Nine Mile Strain ATCC VR-616, Francisella tularensis ATCC 15482. Stored at −30° C.

Bartonella henselae ATCC 49882, Bartonella quintana ATCC 49793, Bartonella clarridgeae (deposited strain, collection: Unite des Rickettsies, Faculté de médecine, Marseille), Bartonella, mass ilae (deposited strain, collection: Unité des Rickettsies, Faculté de médecine, Marseille). Stored at 4° C.
Detection antibody: goat anti-human immunoglobulin IGH (Fluoline-H, bioMérieux sa, Marcy l'Etoile, France).

4) Preparation of Reagents

The slides must be degreased with methyl alcohol for at least 2 h and ideally overnight.
Preparation of antigens:
   After thawing and bringing to room temperature, dilution to 1/2 in sterile PBS (storage at 4° C.). The quality of the batch of antigen is previously verified by testing with a control serum (i.e. with a known antibody concentration) to determine the optimum dilution for this antigen's use.

5) Slide Preparation
The antigens are deposited on the degreased slide spots using a different pen for each antigen. These pens are rinsed, wiped and dried after each use.
The first screening slide must comprise the following antigens, at all times deposited in the same position starting from the top and proceeding clockwise: Staphylococcus aureus, Coxiella burnetii, Rickettsia slovaca, Rickettsia conorii, Rickettsia typhi. The slides are fixed in acetone for 10 nm.
The second screening slide must comprise the following antigens, at all times deposited in the same position starting from the top and proceeding clockwise: Staphylococcus aureus, Bartonella henselae, Bartonella clarridgeae, Bartonella quintana, Francisella tularensis et B. massiliae to the medium. The slides are fixed in methyl alcohol for 10 nm.
Reminder: microscope reading will be in reverse.

6) Dilution of the Sera to be Tested:

The sera must be decomplemented, numbered and computer recorded.

Preparation of Dilutions.

Three dilutions 1/25, 1/50, 1/100 are systematically made in milk PBS (for LCR dilution 1/2, 1/4, 1/8, for tick bite 1/8)

Depositing the Dilutions:
30 μl of each of the 3 dilutions from each patient, one under the other.
The first spot on the slide is reserved for the positive control formed of a pool of positive sera known for each of the tested antigens.
The last spot on the slide is reserved for the negative control (milk PBS).

Leave to incubate 30 minutes at 37° C. in a humid atmosphere.

7) Rinsing of Slides

The slides are washed with a PBS spray and in baths of PBS tween, then in distilled water;

8) Depositing the Immunoglobulins

Preparation: total fluorescent IgH to be diluted in milk PBS with a drop of Evans blue at 1/400 (provided the dilution has been tested on opening the bottle following the procedure developed under point no4). 30 μl are deposited on each spot. Leave to incubate for 30 minutes at 37° C. in a humid atmosphere then rinse following the same procedure as described above.

9) Slide Mounting

3 drops of Fluoprep® are deposited on the dry slides. A glass cover is positioned keeping it still, and the Fluoprep® is allowed to diffuse over the entire slide. The slides are stored away from light until the reading step.

10) Reading

This is performed on the fluorescence microscope. Reading under the microscope is reversed in relation to the deposit. All positive readings are to be recorded on the worksheet.

2. Results

A serum series was collected from blood donors (general population controls) (n=100) and from patients suffering from an endocarditis (n=400), cat scratch disease due to the bacterium Bartonella henselae (n=50), an acute infection with Coxiella burnetii (acute Q fever) (n=50), or various infections characterized by a non-specific polyclonal increase in serum immunoglobulin levels (infection with Epstein-Barr virus, H.I.V. infection, C.M.V. infection) (n=50).

This serological test showed an anti-Staphylococcus aureus antibody titre of 1:400 in 100% of patients suffering from an endocarditis, and a titre of 1:200 in 100% of the other patients and in the blood donors.

A control operation was performed on 10 tests in which the sample to be tested only contained the conjugated detection immunoglobulin and in which the patient serum had been deliberately omitted. No fluorescence was detected in these 10 samples confirming the absence of reaction by goat immunoglobulin with protein A. These results show that all human serum incorporated in an indirect immunofluorescence reaction at a dilution of ≦:1:200 shows a reaction against Staphylococcus aureus; that this reaction is not deteriorated by infectious pathologies, including infectious pathologies which induce polyclonal hypergammaglobulinaemia and hence strongly modify the level and quality of human serum immunoglobulins; and that the absence of human serum translates as absence of fluorescence.

For the results given in FIGS. 1A, 1B and 1C, the test was performed against three antigens, S. aureus ATCC 29 213 in the centre of the slide, R. conorii ATCC VR-141 on the left of the slide, and B. henselae ATCC 49882 on the right of the slide.
FIG. 1A=with no human serum,
FIG. 1B=with human serum positive for R. conorii,
FIG. 1C=with human serum positive for B. henselae.

The omission of human serum leads to the absence of fluorescence (FIG. 1A), the addition of serum systematically leads to fluorescence against S. aureus (FIGS. 1B and 1C).

1. In vitro serological diagnosis method in which, in a sample to be tested, the presence is detected of antibodies specific to an infectious microbial agent, characterized in that it is controlled that said sample to be tested contains a human serum by detecting whether human immunoglobulins react with an antigen containing protein A from a Staphylococcus aureus bacterium.

2. Serological diagnosis method as in claim 1, characterized in that:
the sample to be tested is caused to react with a first antigen (Ag₁) containing protein A, preferably all or part of a Staphylococcus aureus bacterium containing protein A, and
the presence is detected of an antigen-antibody reaction product (Ag₁-Ac₁) in which the antibody (Ac₁) is a human immunoglobulin, by causing said reaction product (Ag₁-Ac₁) to react with a detection substance which is a substance reacting with a human immunoglobulin and not reacting with said first antigen (Ag₁).

3. Serological diagnosis method as in claim 1, characterized in that the following steps are performed, in which:
a) on a solid-substrate-are-deposited said first antigen containing-protein A (Ag₁), and at least one second antigen (Ag₂) which is characteristic of a microbial infectious agent (Ag₂), and
b) the said first antigen (Ag₂) and second (Ag₂) antigen(s) are caused to react with a sample to be tested, and
c) it is detected whether a human immunoglobulin (Ac₁) reacts with said first antigen (Ag₁) by causing the reaction product (Ag₁-Ac₁) to react with a secondary detection antibody (Ac₂) which is a labelled anti-human immunoglobulin which does not react with protein A.

4. Serological diagnosis method as in claim 1, characterized in that said first antigen is a whole Staphylococcus aureus bacterium containing protein A.

5. Serological diagnosis method as in claim 1, characterized in that the presence is detected of a said reaction product (Ag₁-Ac₁) with an anti-human immunoglobulin (Ac₂) which is an immunoglobulin of animal origin, preferably goat or chick immunoglobulin.

6. Serological diagnosis method as in claim 1, characterized in that the presence is detected of a reaction product of said first antigen (Ag₁) with a human immunoglobulin (Ac₁) using a substance labelled by fluorescent marking, in particular an anti-human immunoglobulin labelled with fluorescein.

7. Serological diagnosis method as in claim 6, characterized in that:
a series of tests is performed at increasing dilutions of the sample to be tested and the detection substance (Ac₂) is applied which is an immunoglobulin conjugated with a fluorescent substance, and
it is verified whether a reaction product (Ag₁-Ac₁-Ac₂) can be detected by fluorescence at a dilution of the sample to be tested of 1/200 or less.

8. Serological diagnosis method as in claim 1, characterized in that said infectious microbial agent consisting of said second antigen is chosen from among micro-organisms containing a bacterium, a virus, a parasite or a fungus.

9. Serological diagnosis method as in claim 8, characterized in that said second antigen (Ag₂) is an intracellular bacterium or a virus.

10. Serological diagnosis method as in claim 8, characterized in that said second antigen is chosen from among bacteria of the genus Rickettsia, Coxiella, Bartonella, Tropheryma, Ehrlichia, Chlamydia, Mycoplasma, Treponema, Borrelia, and Leptospira.

11. Serological diagnosis method as in claim 10, characterized in that said second antigen corresponding to the infectious microbial agent is a bacterium responsible for endocarditis.

12. Serological diagnosis method as in claim 9, characterized in that said second antigen corresponding to said infectious microbial agent is a viral antigen chosen from among the H.I.V., C.M.V. or Epstein-Barr viruses.

13. Diagnosis kit which can be used to implement the method as in claim 1, characterized in that it includes at least one positive control controlling inclusion of a human serum in the sample to be tested comprising a said first antigen containing protein A (Ag₁) and reagents enabling the detection of the presence of a reaction product of said first antigen with a human immunoglobulin (Ac₁).

14. Diagnosis kit as in claim 13, characterized in that it includes
a solid substrate on which a said first protein A-containing antigen has been deposited (Ag₁) and a said second antigen corresponding to an infectious microbial agent (Ag₂) to be detected, and
a detection substance (Ac₁) to detect a reaction product of said first antigen with a human immunoglobulin containing a labelled anti-human immunoglobulin which is a goat or chick immunoglobulin labelled with fluorescent marking.
